(19) United States
(12) Patent Application Publication
(10) Pub. No.: US 2016/0249997 A1
(43) Pub. Date: Sep. 1, 2016

(54) FORCE-ASSISTING CALIBRATION DEVICE

(71) Applicant: BRAINLAB AG, Feldkirchen (DE)

(72) Inventors: Thomas Feilkas, Kirchseeon (DE); Christian Lechner, Turkenfeld (DE); Johanna Woeste, Munich (DE)

(21) Appl. No.: 15/032,431

(22) PCT Filed: Jan. 14, 2014
(86) PCT No.: PCT/EP2014/050575

§ 371 Date: Apr. 27, 2016

(51) Int. Cl. A61B 90/00; A61B 34/20; A61B 17/00

(52) U.S. Cl. CPC A61B 90/06; A61B 17/00; A61B 90/39; A61B 34/20; A61B 2017/00725; A61B 2017/00876; A61B 2034/2055; A61B 2090/3925; A61B 2034/2051

(57) ABSTRACT

A calibration device for calibrating a medical instrument, comprising at least one supporting surface which is configured to provide support for a medical instrument which is brought into contact with at least one of the one or more supporting surfaces for a calibration procedure, wherein the calibration device comprises means which exert at least one force on the medical instrument, the at least one force being directed towards the at least one of the one or more supporting surfaces.

The invention relates to the general technical field of calibration devices for calibrating surgical instruments used in conjunction with various medical procedures.

In order to be used in image-guided surgery, instruments have to be calibrated, so that their precise size, geometry and configuration is known while the surgical procedure is being performed. In particular, the spatial position of the instrument's functional section, such as for example the instrument tip, must be determined relative to at least one tracking marker which is assigned to the instrument and allows the instrument to be tracked by means of a surgical tracking system.

WO 02/061371 A1 shows a calibration device having a V-shaped groove or channel into which the cylindrical shaft of an instrument to be calibrated is inserted, wherein the tip of the instrument rests against a stop provided in the groove. In order to ensure that the instrument is positioned precisely within the groove during the calibration procedure, a self-actuating tool holder comprising a spring-biased ball bearing is positioned above the groove, such that the tool is securely held in the groove by the spring-biased ball bearing. The direction of the spring force acting on the instrument shaft points exactly towards the apex of the groove, thereby passing both of the flat surface areas extending from the apex on both sides. The applied spring force therefore has to hold the instrument shaft firmly against both of the surface areas of the V-shaped groove at a relatively small angle between the direction of the spring force and each of the surface areas, such that it can be difficult to hold the instrument shaft firmly on the two surface areas. It is therefore quite easy for the instrument shaft to tilt within the V-shaped groove, thus precluding an exact and precise calibration procedure.

It is the object of the present invention to provide a calibration device which allows a medical instrument to be calibrated in an efficient and reliable manner.

This problem is solved by the subject-matter of any appended independent claim. Advantages, advantageous features, advantageous embodiments and advantageous aspects of the present invention are disclosed in the following and contained in the subject-matter of the dependent claims. Different advantageous features can be combined in accordance with the invention wherever technically expedient and feasible.

The calibration device according to the invention comprises at least one supporting surface which is configured to provide support for a medical instrument which is brought into contact with at least one of the one or more supporting surfaces for a calibration procedure, wherein the calibration device comprises means which exert at least one force on the medical instrument, the at least one force being directed towards the at least one of the one or more supporting surfaces.

In other words, the calibration device according to the invention comprises one or more supporting surfaces which can be used to ensure that the instrument to be calibrated is positioned precisely relative to the calibration device by a physical contact between the instrument and the at least one supporting surface. It is necessary to maintain this physical contact during a calibration procedure which may include moving the instrument relative to the calibration device, for example about the axis of a longitudinal instrument shaft. In order to aid in maintaining this physical contact and therefore also the exact placement of the instrument during the calibration procedure, the calibration device according to the invention comprises means which exert at least one force on the medical instrument, in particular on the sections of the instrument which lie nearest the respective supporting surface. Each of the forces is assigned to a respective supporting surface and is directed towards the respective supporting surface, so as to maintain the physical contact between each of the respective supporting surfaces and the instrument to be calibrated. The angle between the direction of said force and the respective supporting surface is preferably between 80° and 100° and more preferably about 90°. For each supporting surface, the corresponding force generated by the force exerting means of the respective supporting surface is directed towards the supporting surface.

In accordance with a preferred embodiment of the present invention, at least one of the one or more supporting surfaces comprises said force exerting means. In other words, the medical instrument which contacts the respective supporting surface does not lie between said force exerting means and the supporting surface. In particular, the respective force exerting means lies on or underneath the respective supporting surface, opposite the medical instrument which contacts the supporting surface. With the force exerting means positioned this way, each of the supporting surfaces “pulls” the instrument towards itself.

The calibration device according to the invention preferably comprises at least two supporting surfaces which are arranged so as to simultaneously support one medical instrument during a calibration procedure. Two such supporting surfaces can for example form a V-shaped groove in order to support a cylindrical instrument shaft, wherein an additional supporting surface can serve as a stop which supports the tip of the cylindrical shaft or instrument. In addition, the at least two supporting surfaces can be configured to allow a rotational movement and/or a translational movement of the medical instrument relative to the calibration device during a calibration procedure. Using this embodiment of the invention comprising three supporting surfaces which form a V-shaped groove and a stop, various instruments comprising longitudinal cylindrical shafts of different diameters can be calibrated in terms of their shaft axis and instrument tip, for example by rotating the instrument shaft around its longitudinal axis within the V-shaped groove, thereby maintaining the contact between the instrument and each of the three supporting surfaces.

In accordance with another preferred embodiment, the force exerting means are configured to restrict the movement of a medical instrument relative to the calibration device to a rotational movement or to a translational movement. Such a configuration prevents any unintended tilting of the instrument relative to the supporting surfaces.

At least one of the one or more supporting surfaces can also be configured to provide one or more forms of contact with the medical instrument, in particular one or more of:
zero-dimensional/punctual contact;
one-dimensional/linear contact; and
two-dimensional/planar contact.

It is generally conceivable for the position of the calibration device to be spatially fixed within the surgical environment, wherein the invariant position of each of the supporting surfaces is known to the tracking system. In accordance with one preferred embodiment of the present invention, however, a preferred calibration device can be freely moved within the surgical environment. If the calibration device is not configured to be registered each time it has been moved, for example by using a pointer instrument to palpate at least three known registration marks provided on the calibration device, then the calibration device preferably comprises at least one tracking marker which allows a medical tracking system to track the calibration device in real time. Such tracking markers can be active or passive optical tracking markers, electromagnetic tracking markers or ultrasound tracking markers which have to exhibit a predetermined spatial relationship with respect to each of the supporting surfaces.

The force exerted on the medical instrument can be one or more of various kinds of forces. The force exerting means can for example comprise electromagnets which can be switched on or off in order to hold the instrument on the supporting surfaces using an electromagnetic force. Suction means which generate a vacuum between the supporting surfaces and the instrument are also conceivable. Alternatively or additionally, an electrostatic force can be exerted on the instrument by the force exerting means.

In accordance with a preferred embodiment of the present invention, the force exerting means exert a magnetic force on the medical instrument. To this end, at least one of the supporting surfaces can comprise a permanent magnet on and/or underneath the supporting surface which then “pulls” the instrument towards the supporting surface. Additionally or alternatively to the permanent magnet, an electromagnet can be provided.

In general, the invention can also provide a force fit between the calibration device and the medical instrument to be calibrated.

The calibration device according to the invention can also comprise a plurality of supporting surfaces which are configured to provide support for different medical instruments. An arrangement of at least two supporting surfaces which can form a V-shaped groove can for example allow a calibration procedure to be performed for instruments comprising a longitudinal cylindrical shaft by turning the instrument about the longitudinal axis of the cylindrical shaft, wherein an additional supporting surface can be provided in order to allow at least one other kind of medical instrument to be calibrated, such as for example a chisel which comprises a blade which can be supported by a flat, angled supporting surface.

In the following, the invention is described with reference to the enclosed figures which represent preferred embodiments of the invention, though without limiting the scope of the invention to the specific features shown in the figures.

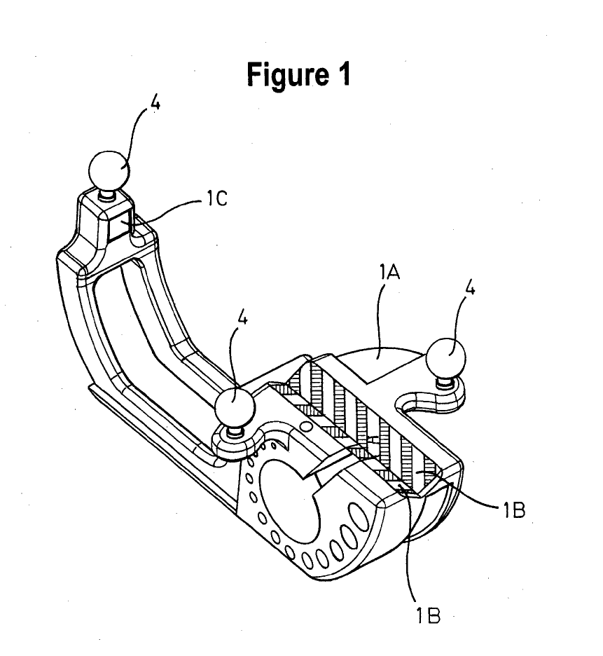

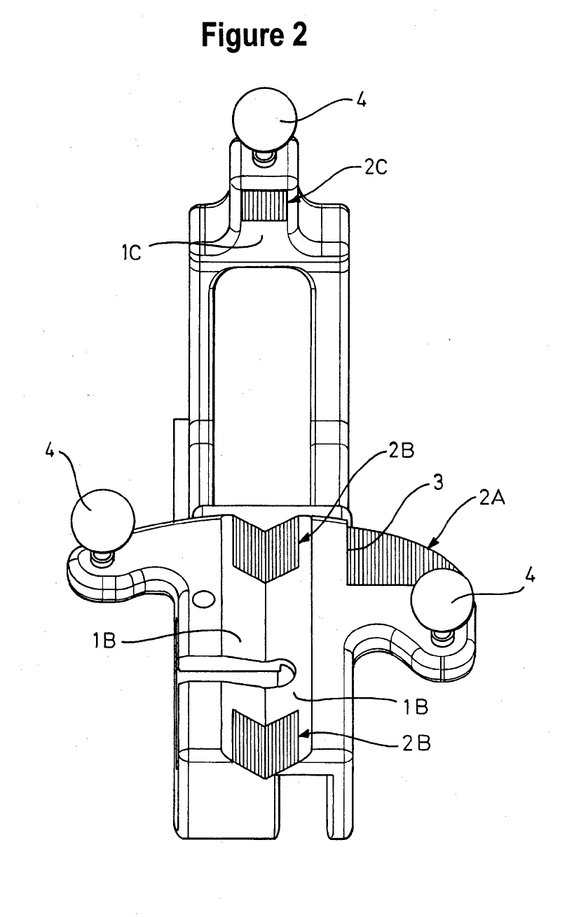

FIGS. 1 and 2 show a preferred embodiment of the calibration device according to the invention.

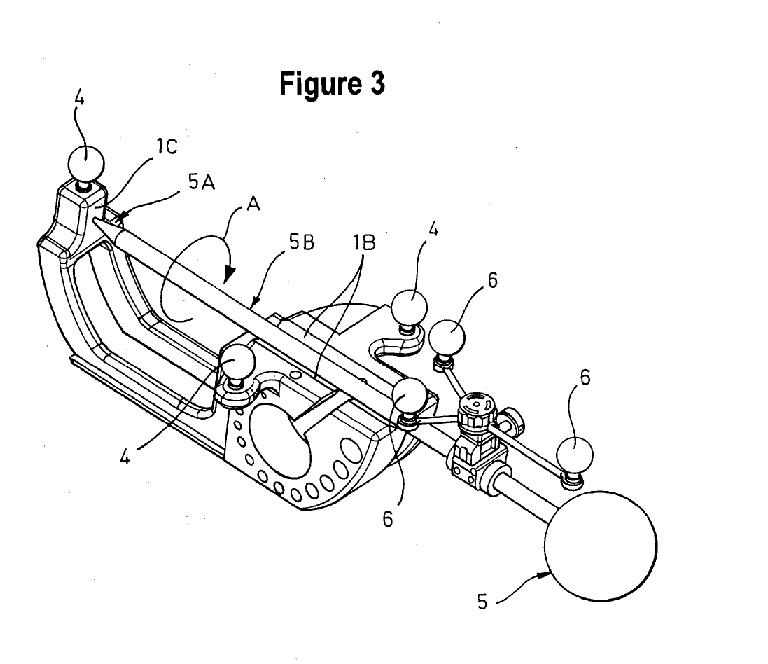

FIG. 3 shows a medical instrument comprising a longitudinal cylindrical shaft which is brought into contact with the calibration instrument in order to perform a calibration procedure.

FIGS. 1 and 2 show a preferred embodiment of the calibration device according to the invention, comprising two arrangements of supporting surfaces 1A to 1C. The first arrangement comprises a single supporting surface 1A which exhibits a plane surface allowing two-dimensional (planar) or at least one-dimensional (linear) contact with an instrument. A permanent magnet 2A (FIG. 2) is also provided on the surface 1A, the magnetic force of which attracts any instrument comprising a ferromagnetic material towards the edge 3 which is formed on the supporting surface 1A and serves as a stop for the flat blade tip of instruments such as chisels. The supporting surfaces 1B and 1C form another arrangement of supporting surfaces which allows rotationally symmetrical tools to be calibrated. Two supporting surfaces 1B, each comprising a permanent magnet 2B, form a V-shaped groove or notch which allows a rotational movement of a rotationally symmetrical instrument which is in one-dimensional or linear contact with each of the two surfaces 1B. The tip of the instrument contacts the supporting surface 1C in a two-dimensional (planar) or at least zero-dimensional (punctual) contact and is pulled towards the supporting surface 1C by another permanent magnet 2C (FIG. 2).

All of the supporting surfaces 1A to 1C are provided on the calibration device in a predetermined spatial relationship with respect to three passive tracking markers 4 which are configured to be tracked by a medical tracking system, so that the spatial position of the calibration device and its supporting surfaces 1A to 1C are known to the medical tracking system.

FIG. 3 shows a calibration procedure for a rotationally symmetrical instrument 5 which comprises a shaft 5B and a tip 5A and is inserted into the V-shaped groove or notch formed by the supporting surfaces 1B, During the calibration procedure, in which the instrument is rotated about its longitudinal axis, the instrument tip 5A contacts the supporting surface 1C and the shaft 5B contacts each of the two supporting surfaces 1B which form the V-shaped groove. As the instrument 5 is rotated within the V-shaped notch (as indicated by the arrow A in FIG. 3), the movement of the tracking markers 6 attached to the instrument is detected by an optical medical tracking system, such that data can be provided from which the longitudinal axis and the diameter of the instrument shaft 5B can be calculated. The position of the instrument tip 5A relative to the tracking markers 6 is similarly determined with the aid of the tracking markers 4.

1-10. (canceled)

11. A calibration device for calibrating a medical instrument, comprising at least one supporting surface providing support for a medical instrument which is brought into contact with at least one of the one or more supporting surfaces for a calibration procedure, wherein the calibration device comprises a force generating component that exerts at least one force on the medical instrument, the at least one force being directed towards the at least one of the one or more supporting surfaces.

12. The calibration device according to claim 11, wherein at least one of the one or more supporting surfaces comprises said force exerting means.

13. The calibration device according to claim 11, wherein at least two supporting surfaces are arranged so as to simultaneously support one medical instrument during a calibration procedure.

14. The calibration device according to claim 13, wherein the at least two supporting surfaces are configured to allow a rotational movement and/or a translational movement of the medical instrument relative to the calibration device during a calibration procedure.

15. The calibration device according to claim 14, wherein the force exerting means are configured to restrict the movement of the medical instrument relative to the calibration device to a rotational movement or to a translational movement.

16. The calibration device according to claim 11, wherein at least one of the one or more supporting surfaces provides one or more forms of contact with the medical instrument the one or more forms of contact being selected from the group consisting of: zero-dimensional/punctual contact; a one-dimensional/linear contact; and/or a two-dimensional/planar contact.

17. The calibration device according to claim 11, comprising at least one tracking marker which exhibits a predetermined spatial relationship relative to each of the one or more supporting surfaces.

18. The calibration device according to claim 11, wherein the force is exerted on the medical instrument by a force fit provided between the calibration device and the medical instrument.

19. The calibration device according to claim 11, wherein the force is exerted on the medical instrument by providing a magnet which exert a magnetic force on the medical instrument.

20. The calibration device according to claim 11, comprising a plurality of supporting surfaces providing support for different medical instruments.
